(19) United States
(12) Patent Application Publication
(10) Pub. No.: US 2013/0218029 A1
(43) Pub. Date: Aug. 22, 2013

(54) SYSTEM AND METHOD FOR ASSESSING RENAL ARTERY NERVE DENSITY

(71) Applicants: Martin Cholette, Acton, CA (US); Gary R. Dulak, Moorpark, CA (US)

(72) Inventors: Martin Cholette, Acton, CA (US); Gary R. Dulak, Moorpark, CA (US)

(73) Assignee: PACESETTER, INC., Sylmar, CA (US)

(21) Appl. No.: 13/398,706

(22) Filed: Feb. 16, 2012

(51) Int. Cl. A61B 5/0205; A61N 5/06; A61N 1/36; A61B 5/05; A61B 6/00

(52) U.S. Cl. 600/480; 600/554; 600/483; 600/479; 607/62; 607/88

(57) ABSTRACT

A system and method is described to map the renal artery prior to an ablation in order to a-priori identify the location of the sympathetic nerves. In specific embodiments, the nerve modulating energy may be electrical or optical.

### FIELD OF THE INVENTION

The present invention relates generally to locating nerves in blood vessels, and more specifically to an assessment method for renal artery nerve density.

### BACKGROUND OF THE INVENTION

Hypertension (HTN), or high blood pressure (HBP), is defined as a consistently elevated blood pressure (BP) greater than or equal to 140 mmHg systolic blood pressure (SBP) and 90 mmHg diastolic blood pressure (DBP). Hypertension is a “silent killer” that is not associated with any symptoms and in 95% of cases (primary hypertension) the specific cause is unknown. In the remaining 5% of patients (secondary hypertension), specific causes including chronic kidney disease, diseases of the adrenal gland, coarctation of the aorta, thyroid dysfunction, alcohol addiction, pregnancy or the use of birth control pills are present. In secondary hypertension, when the root cause is treated, blood pressure usually returns to normal.

Hypertension is a disease that affects 74.5 million patients in the US with 24% or 17.7 million patients classified as uncontrolled hypertensive patients. Of these 17.7 million US patients, 27% of them are resistant to drug therapy without any secondary causes. This equates to 4.8 million patients in the US and an estimated 12.4 million patients outside of the US for a total of 17.2 million patients worldwide. Needless to say, there is a need for additional therapeutic options for this class of unsuccessfully treated patients.

It is generally accepted that the causes of hypertension are multi-factorial, with a significant factor being the chronic hyper-activation of the sympathetic nervous system (SNS), especially the renal sympathetic nerves. Renal sympathetic efferent and afferent nerves, which lie in the wall of the renal artery, have been recognized as a critical factor in the initiation and maintenance of systemic hypertension. Renal arteries, like all major blood vessels, are innervated by perivascular sympathetic nerves that traverse the length of the arteries. The perivascular nerves consist of a network of axons, terminals, and varicosities, which are distributed mostly in the medial-adventitial and adventitial layers of the arterial wall.

Signals coming in to the kidney travel along efferent nerve pathways and influence renal blood flow, trigger fluid retention, and activate the renin-angiotensin-aldosterone system cascade. Renin is a precursor to the production of angiotensin II, which is a potent vasoconstrictor, while aldosterone regulates how the kidneys process and retain sodium. All of these mechanisms serve to increase blood pressure. Signals coming out of the kidney travel along afferent nerve pathways integrated within the central nervous system, and lead to increased systemic sympathetic nerve activation. Chronic over-activation can result in vascular and myocardial hypertrophy and insulin resistance, causing heart failure and kidney disease.

Previous clinical studies have documented that denervating the kidney has a positive effect for both hypertension and heart failure patients. Journal articles published as early as 1936 review surgical procedures called either sympathectomy or splanchnicectomy, to treat severe hypertension. A 1953 JAMA article by Smithwick et al. presented the results of 1,266 cases of surgical denervation to treat hypertension. The results included radiographic evidence of hearts that had remodeled after the surgery, while also showing significant blood pressure declines. Additional articles published in 1955 and 1964 demonstrated that the concept of using renal denervation to lower blood pressure and treat heart failure was viable. However, given the highly invasive and traumatic nature of the procedure and the advent of more effective antihypertensive agents, the procedure was not widely employed.

More recently, catheter ablation has been used for renal sympathetic denervation. Renal denervation is a method whereby amplified sympathetic activities are suppressed to treat hypertension or other cardiovascular disorders and chronic renal diseases. The objective of renal denervation is to neutralize the effect of renal sympathetic system which is involved in arterial hypertension. The renal sympathetic efferent and afferent nerves lie within and immediately adjacent to the wall of the renal artery. Energy is delivered via a catheter to ablate the renal nerves in the right and left renal arteries in order to disrupt the chronic activation process. As expected, early results appear both to confirm the important role of renal sympathetic nerves in resistant hypertension and to suggest that renal sympathetic denervation could be of therapeutic benefit in this patient population.

In clinical studies, therapeutic renal sympathetic denervation has produced predictable, significant, and sustained reductions in blood pressure in patients with resistant hypertension. Catheters are flexible, tubular devices that are widely used by physicians performing medical procedures to gain access into interior regions of the body. A catheter device can be used for ablating renal sympathetic nerves in therapeutic renal sympathetic denervation to achieve reductions of blood pressure in patients suffering from renal sympathetic hyperactivity associated with hypertension and its progression. Renal artery ablation for afferent and efferent denervation has been shown to substantially reduce hypertension.

The efficacy of renal denervation for the treatment of hypertension is related to the degree of renal denervation achieved. The distribution of sympathetic nerves surrounding the renal artery is highly variable. This degree of variability poses a particular challenge to the clinician trying to ablate the nerves. It is not known during the procedure whether a high enough degree of denervation has occurred. Prior denervation procedures assess the degree of denervation after the ablation procedure; i.e. the ablation is performed “blind”, and the degree of denervation is assessed after the fact.

### SUMMARY OF THE INVENTION

Embodiments of the invention provide a method and system to map the renal artery prior to the ablation in order to a-priori identify the location of the sympathetic nerves. This method then allows the clinician to target the ablation procedure, and maximize the degree of denervation. In specific embodiments, one or more parameters are monitored to assess the nerve density in different areas of the renal artery or similar blood vessel.

In accordance with an aspect of the present invention, a method comprises: introducing a catheter having a nerve modulating device into a blood vessel of a patient; applying nerve modulating energy, using the nerve modulating device in the blood vessel, to different areas of the blood vessel; monitoring a parameter of afferent stimulation of the patient before applying the nerve modulating energy; monitoring the parameter of afferent stimulation upon applying the nerve modulating energy to each of the different areas of the blood vessel; and assessing nerve density in the different areas based on the monitored parameter.

In some embodiments, monitoring a parameter of afferent stimulation before applying the nerve modulating energy comprises monitoring a heart rate (HR) and a systolic blood pressure (SysBP) of the patient before applying the nerve modulating energy. Monitoring the parameter of afferent stimulation upon applying the nerve modulating energy comprises monitoring the HR and the SysBP upon applying the nerve modulating energy to the different areas of the blood vessel. Assessing nerve density comprises calculating, for each of the different areas, an innervation index which is Delta HR*Delta SysBP, where Delta HR is a change in the HR and Delta SysBP is a change in the SysBP, upon applying the nerve modulating energy. An increase in value of the innervation index corresponds to an increase in the nerve density.

In specific embodiments, the method further comprises generating an innervation index map of the innervation index as a function of the different areas of the blood vessel; and superimposing the innervation index map with an anatomical image of the blood vessel to provide a superimposed map that shows varying levels of innervation indicative of different nerve densities, including one or more peak innervation levels, in the different areas of the blood vessel. The method further comprises applying denervating energy to one or more target areas of the blood vessel based on the superimposed map, the one or more target areas having relatively higher levels of innervation indicative of relatively higher nerve densities. The denervating energy is applied to the one or more target areas of the blood vessel that include the one or more peak innervation levels.

In accordance with another aspect of the invention, a method comprises: introducing a catheter having a nerve modulating device into a blood vessel of a patient; applying a nerve modulating electrical field, using the nerve modulating device in the blood vessel, to different areas of the blood vessel; monitoring a heart rate of the patient before applying the nerve modulating electrical field; monitoring the heart rate upon applying the nerve modulating electrical field to each of the different areas of the blood vessel; and assessing nerve density in the different areas based on the monitored heart rate. An increase in value of the heart rate corresponds to an increase in the nerve density.

In some embodiments, applying the nerve modulating electrical field to different areas of the blood vessel includes moving the nerve modulating device to different axial positions along a length of the blood vessel and orienting the nerve modulating device toward different circumferential positions around a circumference of the blood vessel. The method further comprises monitoring a systolic blood pressure (SysBP) of the patient before applying the nerve modulating electrical field; and monitoring the SysBP upon applying the nerve modulating electrical field to the different areas of the blood vessel. Assessing nerve density comprises calculating, for each of the different areas, an innervation index which is Delta HR*Delta SysBP, where Delta HR is a change in heart rate (HR) and Delta SysBP is a change in the SysBP, upon applying the nerve modulating electrical field.

In specific embodiments, the method further comprises generating an innervation index map of the innervation index as a function of the different areas of the blood vessel; and superimposing the innervation index map with an anatomical image of the blood vessel to provide a superimposed map that shows varying levels of innervation indicative of different nerve densities, including one or more peak innervation levels, in the different areas of the blood vessel. The method further comprises applying denervating energy to one or more target areas of the blood vessel based on the superimposed map, the one or more target areas having relatively higher levels of innervation indicative of relatively higher nerve densities. The denervating energy is applied to the one or more target areas of the blood vessel that include the one or more peak innervation levels.

In accordance with another aspect of this invention, a method comprises: introducing a catheter having an optical emission port into a blood vessel of a patient; emitting a nerve modulating optical beam from the catheter through the optical emission port; directing the optical beam to different areas of the blood vessel; monitoring a parameter of afferent stimulation of the patient before emitting the optical beam; monitoring the parameter of afferent stimulation upon directing the optical beam to each of the different areas of the blood vessel; and assessing nerve density in the different areas based on the monitored parameter.

In some embodiments, the parameter of afferent stimulation is related to at least one of inotropic effect or dromotropic effect of a heart of the patient. The parameter of afferent stimulation is selected from the group consisting of a heart rate of the patient and a blood pressure of the blood vessel; and an increase in value of the parameter corresponds to an increase in the nerve density. The optical beam is a low-intensity, pulsed infrared light beam. Directing the optical beam to different areas of the blood vessel includes moving the optical emission port to different axial positions along a length of the blood vessel and orienting the optical emission port toward different circumferential positions around a circumference of the blood vessel.

In specific embodiments, monitoring a parameter of afferent stimulation before applying the nerve modulating energy comprises monitoring a heart rate (FIR) and a systolic blood pressure (SysBP) of the patient before emitting the optical beam. Monitoring the parameter of afferent stimulation upon applying the nerve modulating energy comprises monitoring the HR and the SysBP upon directing the optical beam to the different areas of the blood vessel. Assessing nerve density comprises calculating, for each of the different areas, an innervation index which is Delta HR*Delta SysBP, where Delta HR is a change in the HR and Delta SysBP is a change in the SysBP, upon directing the optical beam.

In some embodiments, the method further comprises generating an innervation index map of the innervation index as a function of the different areas of the blood vessel; and superimposing the innervation index map with an anatomical image of the blood vessel to provide a superimposed map that shows varying levels of innervation indicative of different nerve densities, including one or more peak innervation levels, in the different areas of the blood vessel. The method further comprises applying denervating energy to one or more target areas of the blood vessel based on the superimposed map, the one or more target areas having relatively higher levels of innervation indicative of relatively higher nerve densities. The denervating energy is applied to the one or more target areas of the blood vessel that include the one or more peak innervation levels.

These and other features and advantages of the present invention will become apparent to those of ordinary skill in the art in view of the following detailed description of the specific embodiments.

### BRIEF DESCRIPTION OF THE DRAWINGS

FIG. 1 is an illustration of sympathetic afferent and efferent nerve fibers coursing along the abdominal aorta and renal artery.

FIG. 2A is a side elevational view of an artery illustrating different axial positions from distal to proximal.

FIG. 2B is an end view of the artery illustrating different circumferential positions around the circumference.

FIG. 3 is a table showing an example of heart rate changes at different locations in response to nerve modulation.

FIG. 4 shows an example of an anatomy and nerve distribution map.

FIG. 5 is an example of a flow diagram illustrating the renal nerve density assessment method.

FIG. 6 is an example of a flow diagram illustrating a method to use the renal nerve density assessment results to guide a denervation procedure.

FIG. 7 is a schematic diagram illustrating an example of a system for nerve density assessment.

FIG. 8A shows an example of a nerve modulating device that employs electrical energy for nerve modulation.

FIG. 8B shows an example of a nerve modulating device that employs optical energy for nerve modulation.

### DETAILED DESCRIPTION OF THE PREFERRED EMBODIMENTS

In the following detailed description of the invention, reference is made to the accompanying drawings which form a part of the disclosure, and in which are shown by way of illustration, and not of limitation, exemplary embodiments by which the invention may be practiced. In the drawings, like numerals describe substantially similar components throughout the several views. Further, it should be noted that while the detailed description provides various exemplary embodiments, as described below and as illustrated in the drawings, the present invention is not limited to the embodiments described and illustrated herein, but can extend to other embodiments, as would be known or as would become known to those skilled in the art. Reference in the specification to “one embodiment”, “this embodiment”, or “these embodiments” means that a particular feature, structure, or characteristic described in connection with the embodiment is included in at least one embodiment of the invention, and the appearances of these phrases in various places in the specification are not necessarily all referring to the same embodiment. Additionally, in the following detailed description, numerous specific details are set forth in order to provide a thorough understanding of the present invention. However, it will be apparent to one of ordinary skill in the art that these specific details may not all be needed to practice the present invention. In other circumstances, well-known structures, materials, circuits, processes and interfaces have not been described in detail, and/or may be illustrated in block diagram form, so as to not unnecessarily obscure the present invention.

In the following description, relative orientation and placement terminology, such as the terms horizontal, vertical, left, right, top and bottom, is used. It will be appreciated that these terms refer to relative directions and placement in a two dimensional layout with respect to a given orientation of the layout. For a different orientation of the layout, different relative orientation and placement terms may be used to describe the same objects or operations.

Furthermore, some portions of the detailed description that follow are presented in terms of algorithms, flow-charts and symbolic representations of operations within a computer. These algorithmic descriptions and symbolic representations are the means used by those skilled in the data processing arts to most effectively convey the essence of their innovations to others skilled in the art. An algorithm is a series of defined steps leading to a desired end state or result which can be represented by a flow chart. In the present invention, the steps carried out require physical manipulations of tangible quantities for achieving a tangible result. Usually, though not necessarily, these quantities take the form of electrical or magnetic signals or instructions capable of being stored, transferred, combined, compared, and otherwise manipulated. It has proven convenient at times, principally for reasons of common usage, to refer to these signals as bits, values, elements, symbols, characters, terms, numbers, instructions, or the like. It should be borne in mind, however, that all of these and similar terms are to be associated with the appropriate physical quantities and are merely convenient labels applied to these quantities. Unless specifically stated otherwise, as apparent from the following discussion, it is appreciated that throughout the description, discussions utilizing terms such as “processing,” “computing,” “calculating,” “determining,” “displaying,” or the like, can include the actions and processes of a computer system or other information processing device that manipulates and transforms data represented as physical (electronic) quantities within the computer system's registers and memories into other data similarly represented as physical quantities within the computer system's memories or registers or other information storage, transmission or display devices.

The present invention also relates to an apparatus for performing the operations herein. This apparatus may be specially constructed for the required purposes, or it may include one or more general-purpose computers selectively activated or reconfigured by one or more computer programs. Such computer programs may be stored in a computer-readable storage medium, such as, but not limited to optical disks, magnetic disks, read-only memories, random access memories, solid state devices and drives, or any other types of media suitable for storing electronic information. The algorithms and displays presented herein are not inherently related to any particular computer or other apparatus. Various general-purpose systems may be used with programs and modules in accordance with the teachings herein, or it may prove convenient to construct a more specialized apparatus to perform desired method steps. In addition, the present invention is not described with reference to any particular programming language. It will be appreciated that a variety of programming languages may be used to implement the teachings of the invention as described herein. The instructions of the programming language(s) may be executed by one or more processing devices, e.g., central processing units (CPUs), processors, or controllers.

Exemplary embodiments of the invention, as will be described in greater detail below, provide apparatuses and methods for assessing renal artery nerve density.

FIG. 1 is an illustration of sympathetic afferent and efferent nerve fibers coursing along the abdominal aorta and renal artery. The efferent fibers terminate in the renal parenchyma and release norepinephrine in response to stimulation. The afferent fibers project to the medulla and hypothalamus via the thoracic spinal tract. Stimulation of the afferent sympathetic fibers in the renal artery induces a central reflex up-regulation of systemic sympathetic tone. This increase in systemic sympathetic tone can be observed via its effects on target organs including the liver, spleen, pancreas, heart, blood vessels, etc. Of particular interest to this invention is the effect on the heart and blood vessels as these can be readily observed. The afferent stimulation of the renal nerve causes an increase in heart rate and blood pressure as well as other inotropic and dromotropic effects. The afferent stimulation parameters such as heart rate and/or blood pressure can be monitored to assess renal artery nerve density in different areas of the renal artery.

The monitoring of parameters relating to afferent stimulation such as an increase in heart rate and blood pressure distinguishes the present approach from the monitoring of parameters relating to efferent stimulation in U.S. Pat. No. 7,653,438, such as an increase in the patient's urine production, a decrease in the patient's rennin secretion, and a decrease in the patient's sodium retention. The monitoring of afferent stimulation parameters is used to map the renal artery prior to renal denervation as opposed to assessing the degree of denervation afterwards.

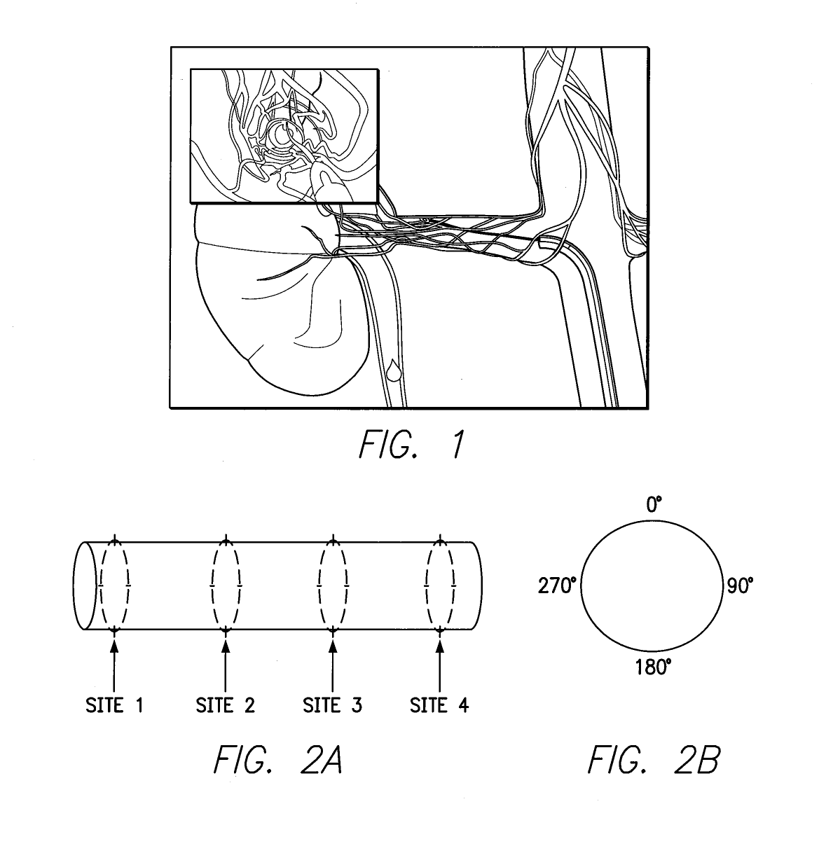

According to embodiments of the present invention, the clinician would advance a catheter in the renal artery and stimulate (electrically, optically, or otherwise) the vessel wall while observing the heart rate. This would be done, for instance, at 0, 90, 180 and 270 degrees (circumferential or angular positions) at various sites in the renal artery going from distal to proximal (axial positions). FIG. 2A is a side elevational view of an artery illustrating different axial positions from distal to proximal. FIG. 2B is an end view of the artery illustrating different circumferential positions around the circumference. The locations (as defined by axial sites and circumferential positions) of maximum innervation will reveal themselves as yielding the most increase in heart rate in response to neuromodulation (i.e., nerve modulation). These maximum innervation locations represent locations having the highest nerve density and will then be the preferred ablation targets for the clinician for achieving denervation.

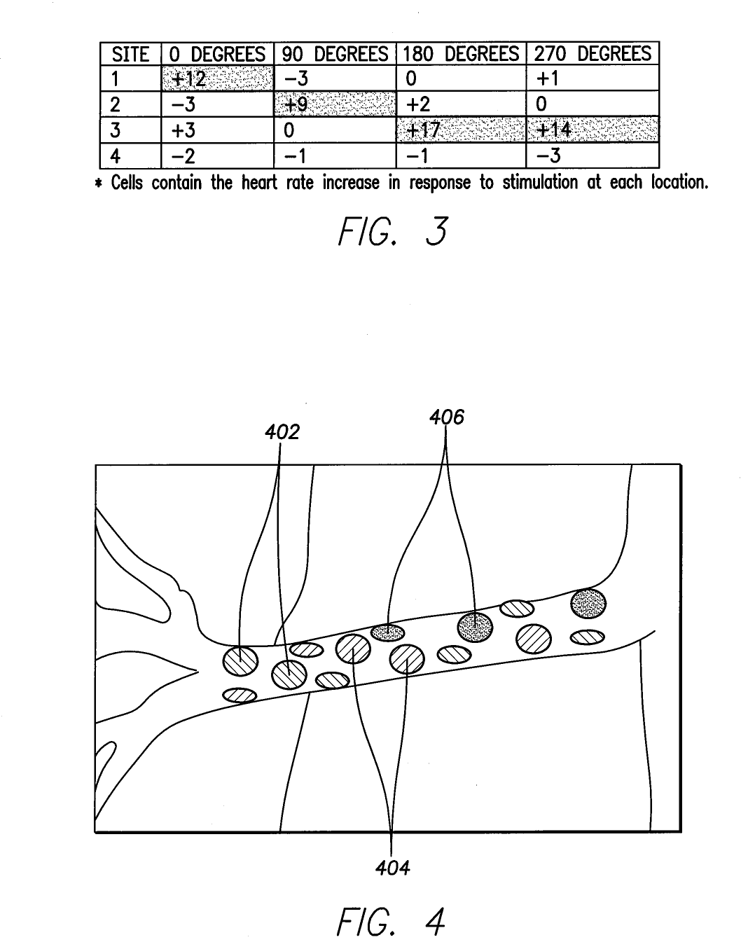

FIG. 3 is a table showing an example of heart rate changes at different locations in response to nerve modulation. In this example, results are obtained at four axial sites (distal to proximal) and four angular positions (0, 90, 180, 270 degrees). The highlighted cells contain the largest heart rate increases in response to neuromodulation at each location, and should be the target of ablation for maximum denervation. The table represents a site response map generated as a result of neuromodulation.

In a further refinement, an “innervation index” can be attributed to each cell instead of a simple parameter such as heart rate. The goal is to make the procedure more sensitive. The innervation index is the product of physiological parameters that change when renal nerves are modulated. An exemplary innervation index is as follows: Innervation Index=Delta HR*Delta SysBP, where Delta HR is a change in the heart rate (HR) (e.g., in beats per minute or BPM) and Delta SysBP is a change in the systolic blood pressure (SysBP) (e.g., in mmHg), upon applying the nerve modulating energy. An increase in value of the innervation index corresponds to an increase in the nerve density.

According to another aspect of the invention, the stimulation and recording of physiological parameters is coupled with a 3D system such as Ensite™ or Mediguide™ available from St. Jude Medical to produce the anatomy and nerve distribution map. FIG. 4 shows an example of an anatomy and nerve distribution map, in which the medium shade (green) 402 indicates high nerve density, the light shade (yellow) 404 indicates moderate nerve density, and the dark shade (red) 406 indicates low nerve density.

The following describes an example of how the integration of the innervation index map and the anatomical image could be performed. Using Mediguide™, the X, Y, Z coordinates and the orientation of the neuromodulation catheter (whether electrical or laser) is continually stored. In a specific embodiment, the X, Y, Z coordinates specify the axial position along the length of the blood vessel, and the orientation specifies the circumferential position around a circumference of the blood vessel. When the renal nerve is modulated, the cardiovascular effect is recorded (e.g., ECG+BP) and the Innervation Index is computed for that specific location (X, Y, Z, Orientation). Repeated neuromodulations at multiple sites in the renal artery yields an “Innervation Index as a function of location” map. The values of Innervation Index on this map can be color coded. The color coded Innervation Index map can be superimposed on an anatomical image (e.g., fluoroscopic image). The clinician has a color coded map of the renal artery showing areas of minimal and maximal innervations (i.e., local valleys and peaks). During denervation (e.g., by ablation) this map can be used to target the delivery of denervation or ablation energy, as the denervation or ablation catheter location can be tracked in real time by the Mediguide™ system and superimposed on the color coded anatomical image.

Exemplary Methods

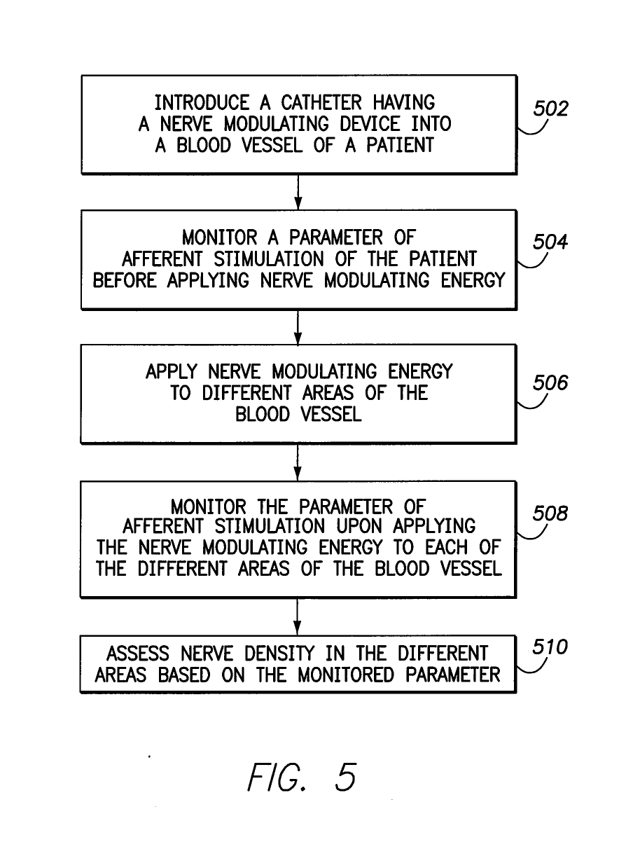

FIG. 5 is an example of a flow diagram illustrating the renal nerve density assessment method. Step 502 is introducing a catheter having a nerve modulating device into a blood vessel of a patient. Step 504 is monitoring a parameter of afferent stimulation of the patient before applying nerve modulating energy. Step 506 is applying nerve modulating energy, using the nerve modulating device in the blood vessel, to different areas of the blood vessel. Step 508 is monitoring the parameter of afferent stimulation upon applying the nerve modulating energy to each of the different areas of the blood vessel. Step 510 is assessing nerve density in the different areas based on the monitored parameter.

In specific embodiments, the parameter of afferent stimulation is related to at least one of inotropic effect or dromotropic effect of a heart of the patient. For example, the parameter of afferent stimulation can be a heart rate of the patient or a blood pressure of the blood vessel. An increase in value of the parameter corresponds to an increase in the nerve density.

The nerve modulating energy may be a nerve modulating electrical field and the parameter being monitored may be the heart rate of the patient. An increase in value of the heart rate corresponds to an increase in the nerve density. To applying the nerve modulating electrical field to different areas of the blood vessel, the nerve modulating device is moved to different axial positions along a length of the blood vessel and oriented toward different circumferential positions around a circumference of the blood vessel. Assessing nerve density comprises calculating, for each of the different areas, an innervation index which is Delta HR*Delta SysBP.

The nerve modulating energy may be a nerve modulating optical beam. The catheter has an optical emission port and the nerve modulating optical beam is emitted from the catheter through the optical emission port and directed to different areas of the blood vessel. In specific embodiments, the optical beam is a low-intensity, pulsed infrared light beam. Directing the optical beam to different areas of the blood vessel includes moving the optical emission port to different axial positions along a length of the blood vessel and orienting the optical emission port toward different circumferential positions around a circumference of the blood vessel.

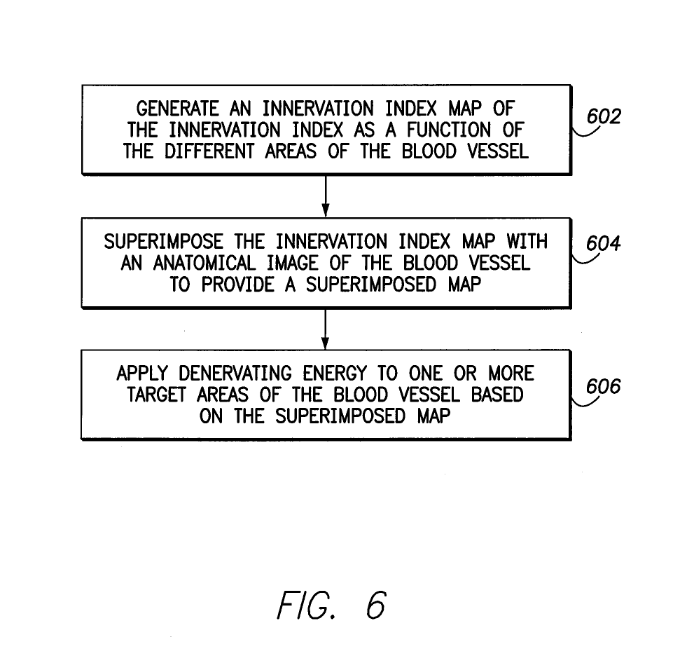

FIG. 6 is an example of a flow diagram illustrating a method to use the renal nerve density assessment results to guide a denervation procedure. Step 602 is generating an innervation index map of the innervation index as a function of the different areas of the blood vessel. Step 604 is superimposing the innervation index map with an anatomical image of the blood vessel to provide a superimposed map that shows varying levels of innervation indicative of different nerve densities, including one or more peak innervation levels, in the different areas of the blood vessel. Step 606 is applying denervating energy to one or more target areas of the blood vessel based on the superimposed map, the one or more target areas having relatively higher levels of innervation indicative of relatively higher nerve densities. Typically, the one or more target areas of the blood vessel include the one or more peak innervation levels.

The denervation typically involves electrical stimulation such as RF ablation, but may employ other methods, including the application of laser, high intensity focused ultrasound (HIFU), cryoablation, or mechanical energy to sever or interrupt conduction of the nerve fibers. In specific embodiments, the same catheter is used for both nerve density assessment and denervation and employs the same type of energy (e.g., electrical energy for both or optical energy for both).

Exemplary Systems

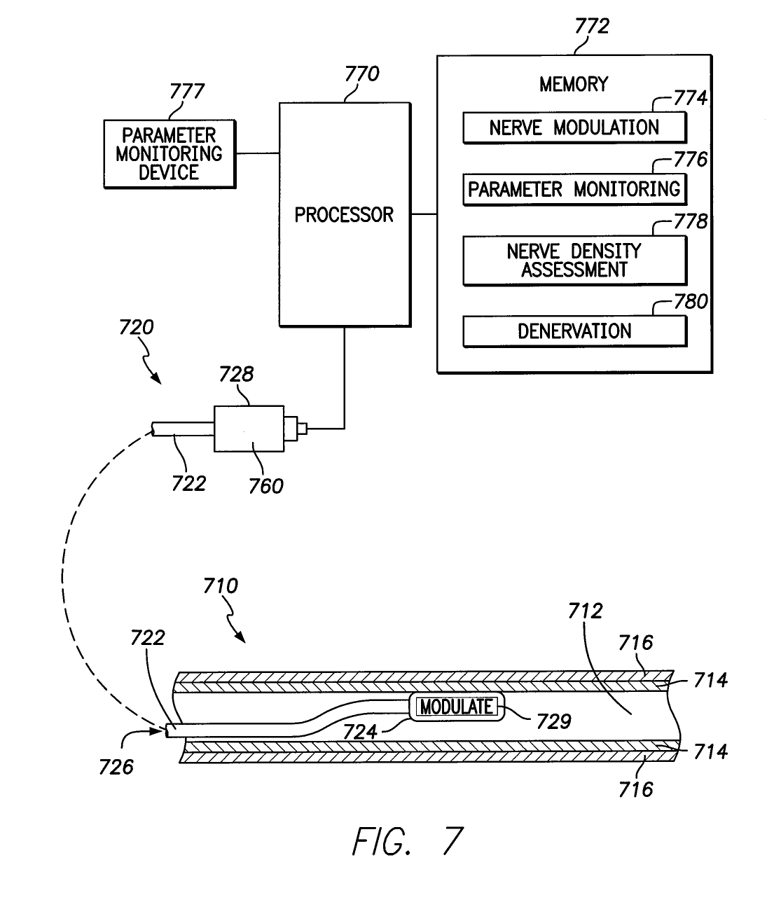

FIG. 7 is a schematic diagram illustrating an example of a system for nerve density assessment. The system 700 includes a catheter device 720 disposed in a vessel 710 of a patient such as a renal artery. The vessel 710 has a vessel wall that defines a lumen 712 such as a blood lumen. In the specific embodiment shown, the vessel wall includes a muscle layer 714 and a nerve layer 716. The catheter 720 has an elongated catheter body 722 extending longitudinally between a proximal end and a distal end along a longitudinal axis. The catheter body 722 includes a distal portion 724 at the distal end, a catheter lumen 726 from the proximal end to the distal end, and typically a handle 728 at the proximal end to manipulate or operate the catheter body 722 and/or other components such as a nerve modulating device, sensors, and the like. In the embodiment shown, the distal portion 724 includes a nerve modulating device 729. In some cases, the same catheter 720 can be used for denervation as well after the nerve density assessment is done. The catheter body 722 preferably has dimensions and flexural properties so as to be deliverable into a renal artery or vein. The catheter body 722 may be introduced into the lumen 712 of the vessel 710 using a guidance sheath or guiding wire (neither shown), or the like.

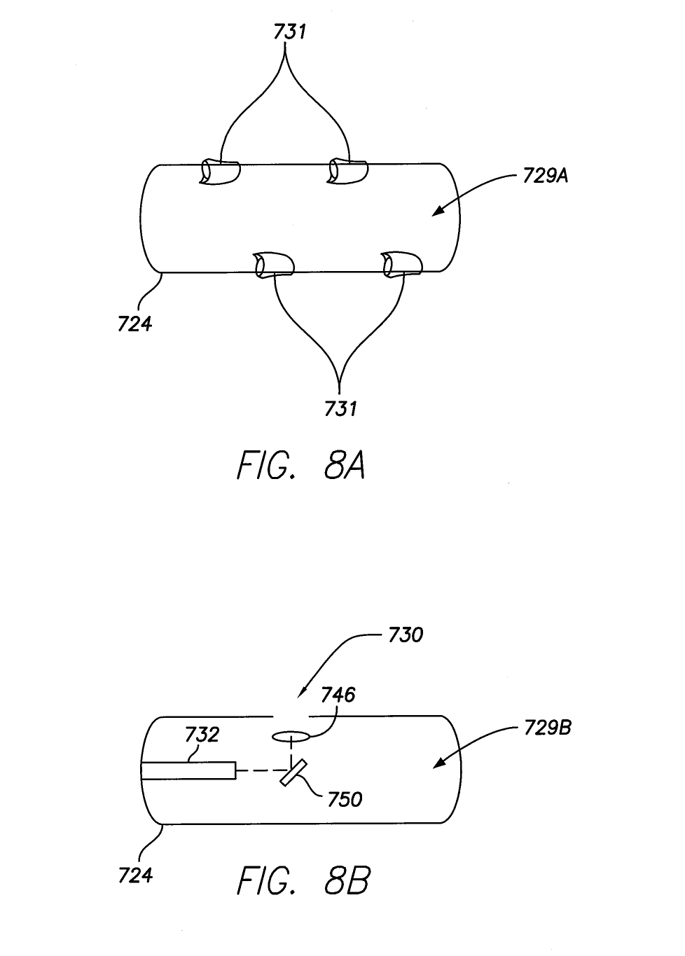

FIG. 8A shows an example of a nerve modulating device 729A that employs electrical energy for nerve modulation. There are various ways of applying electrical energy for nerve modulation. In the example shown, the nerve modulating device 729A has one or more electrodes 731 to apply a nerve modulating electrical field to different areas of the vessel 710.

FIG. 8B shows an example of a nerve modulating device 729B that employs optical energy for nerve modulation. There are various ways of applying optical energy for nerve modulation. In the example shown, the nerve modulating device 729B includes an optical emission port 730 to emit an optical beam outwardly from the distal portion 724. An optical energy delivery conduit 732 extends through the catheter lumen 726 to the optical emission port 730 to deliver optical energy to the optical emission port 730 to produce the emitted optical beam. The optical emission port 730 is capable of delivering the emitted optical beam with sufficient intensity to a depth into the vessel wall of the vessel 710 sufficient to stimulate the nerves but not enough to cause denervation (e.g., to ablate at least one nerve, or to cause tissue removal and physically sever at least one nerve associated with the vessel wall at the depth within that depth range). The optical emission port 730 may be placed in a space which is flushed with blood, such as a blood lumen 712. An optical lens 746 may be provided to focus the optical beam into the vessel wall. The optical energy delivery conduit 732 may include one or more optical fibers. The optical fiber(s) may be bent or include a curved portion from the longitudinal direction to the lateral direction to deliver optical energy to the optical emission port 730. Alternatively, an optical beam redirector 750 is provided to redirect the optical energy from the optical energy delivery conduit 732 to the optical emission port 730 in a direction substantially lateral to the longitudinal axis of the catheter body 722, as seen in FIG. 8B. Examples of the redirector 750 include an optical mirror, reflector, refractor, or prism. The optical redirector 750 may have a reflective coating optimized for the wavelength of the optical energy. The optical energy may be laser energy, LED energy, or the like. In some embodiments, the optical energy for nerve modulation but not denervation is provided by a pulsed, low-energy infrared laser light. To avoid denervation or other damage to the nerves (hence low-energy), stimulation and damage thresholds can be determined as a function of wavelength using a tunable free electron laser source (e.g., λ=2 to 10 micron) and a solid state holmium:YAG laser (e.g., λ=2.12 micron). Threshold radiant exposure required for stimulation varies with wavelength and can be determined by a person of skill in the art (e.g., from about 0.312 J/cm² for 3 micron λ to about 1.22 J/cm² for 2.1 micron λ).

As seen in FIG. 7, a control member 760 is provided to control the electrode(s) 731 of FIG. 8A or the emitted optical beam of FIG. 8B. The control member 760 may be provided at or near the proximal end of the catheter 720. One part of the control member 760 may be used to control the electrical energy delivered to the electrode(s) 731 or the optical energy delivered to the optical emission port 730. Another part may be used to control the placement and movement of the optical emission port 730 or the electrode(s) 731 by manipulating the catheter body 722 and the distal portion 724. The control member 760 may produce manual rotation of the whole catheter 720, autorotation, selective manual tilting of a mirror/prism, selective auto tilting of a mirror/prism, etc. One preferred automated approach has auto rotate around a rotation angle and auto translate along the vessel length of the vessel 710. The control member 760 causes the nerve modulating device 729 (electrical field or emitted optical beam) to be steered, directed, or focused to different parts of the vessel 710, progressively, incrementally, continuously, or otherwise.

In some cases, the same catheter is used for denervation as well as nerve modulation to assess nerve density. The denervation typically involves electrical stimulation such as RF ablation, but may employ other methods, including the application of laser, high intensity focused ultrasound (HIFU), cryoablation, or mechanical energy to sever or interrupt conduction of the nerve fibers. In specific embodiments, the same catheter is used for both nerve density assessment and denervation and employs the same type of energy (e.g., electrical energy for both or optical energy for both). The electrical energy for denervation will be higher than that for nerve modulation to assess nerve density, and RF ablation may be employed. For optical energy, denervation may be achieved with higher power or a different wavelength such as one in the ultraviolet wavelength range. An example is optical energy produced by an Excimer laser. As such, the nerve modulating devices 729A, 729B may be used also for denervation by adjusting the energy source and/or power level. Alternatively, a separate denervation device may be provided in the distal portion 724 of the catheter 720.

The catheter 720 is operated under the control of a processor 770. The processor 770 has circuitries and/or executes software modules stored in memory 772 in order to control the various components of the catheter 720 for nerve modulation and nerve density assessment, and denervation if the same catheter is used for denervation as well. For illustrative purposes, FIG. 7 shows a nerve modulation module 774 to control the nerve modulating device 729 for modulating nerves in different areas of the vessel 710, a parameter monitoring module 776 to control a parameter monitoring device 777 for monitoring one or more parameters of afferent stimulation of the patient, a nerve density assessment module 778 to assess nerve density of the different areas of the vessel 710 based on the nerve modulation and monitored parameter(s), and a denervation module 780 to control the denervation device for denervation of the vessel 710 based on the results of the nerve density assessment. FIG. 7 omits the various circuitries and modules that may be employed including, for example, pulse generation, signal control, switch control, filtering, signal averaging, and the like. The parameter monitoring device 777 may be one or more external devices or one or more implantable devices implanted on the patient, for monitoring afferent stimulation parameters such as heart rate, blood pressure, and the like. Communication between the devices/modules can be wired or wireless.

The system in FIG. 7 can be used to carry out the nerve density assessment procedure of FIG. 5 and the denervation procedure of FIG. 6. For example, the nerve modulation module 774 is used for step 506, the parameter monitoring module 776 is used for steps 504 and 508, the nerve density assessment module 778 is used for step 510, and the nerve denervation module 780 is used for step 606. A separate mapping module may be provided for steps 602 and 604, or the nerve denervation module 780 may be used to perform steps 602 and 604 as well.

In the description, numerous details are set forth for purposes of explanation in order to provide a thorough understanding of the present invention. However, it will be apparent to one skilled in the art that not all of these specific details are required in order to practice the present invention. It is also noted that the invention may be described as a process, which is usually depicted as a flowchart, a flow diagram, a structure diagram, or a block diagram. Although a flowchart may describe the operations as a sequential process, many of the operations can be performed in parallel or concurrently. In addition, the order of the operations may be re-arranged.

From the foregoing, it will be apparent that the invention provides methods, apparatuses and programs stored on computer readable media for nerve density assessment. Additionally, while specific embodiments have been illustrated and described in this specification, those of ordinary skill in the art appreciate that any arrangement that is calculated to achieve the same purpose may be substituted for the specific embodiments disclosed. This disclosure is intended to cover any and all adaptations or variations of the present invention, and it is to be understood that the terms used in the following claims should not be construed to limit the invention to the specific embodiments disclosed in the specification. Rather, the scope of the invention is to be determined entirely by the following claims, which are to be construed in accordance with the established doctrines of claim interpretation, along with the full range of equivalents to which such claims are entitled.

What is claimed is:

1. A method comprising:
introducing a catheter having a nerve modulating device into a blood vessel of a patient;
applying nerve modulating energy, using the nerve modulating device in the blood vessel, to different areas of the blood vessel;
monitoring a parameter of afferent stimulation of the patient before applying the nerve modulating energy;
monitoring the parameter of afferent stimulation upon applying the nerve modulating energy to each of the different areas of the blood vessel; and
assessing nerve density in the different areas based on the monitored parameter.

2. The method of claim 1,
wherein monitoring a parameter of afferent stimulation before applying the nerve modulating energy comprises monitoring a heart rate (HR) and a systolic blood pressure (SysBP) of the patient before applying the nerve modulating energy; and
wherein monitoring the parameter of afferent stimulation upon applying the nerve modulating energy comprises monitoring the HR and the SysBP upon applying the nerve modulating energy to the different areas of the blood vessel;
wherein assessing nerve density comprises calculating, for each of the different areas, an innervation index which is Delta HR*Delta SysBP, where Delta HR is a change in the HR and Delta SysBP is a change in the SysBP, upon applying the nerve modulating energy; and
wherein an increase in value of the innervation index corresponds to an increase in the nerve density.

3. The method of claim 2, further comprising:
generating an innervation index map of the innervation index as a function of the different areas of the blood vessel; and
superimposing the innervation index map with an anatomical image of the blood vessel to provide a superimposed map that shows varying levels of innervation indicative of different nerve densities, including one or more peak innervation levels, in the different areas of the blood vessel.

4. The method of claim 3, further comprising:
applying denervating energy to one or more target areas of the blood vessel based on the superimposed map, the one or more target areas having relatively higher levels of innervation indicative of relatively higher nerve densities.

5. The method of claim 4,
wherein the denervating energy is applied to the one or more target areas of the blood vessel that include the one or more peak innervation levels.

6. A method comprising:
introducing a catheter having a nerve modulating device into a blood vessel of a patient;
applying a nerve modulating electrical field, using the nerve modulating device in the blood vessel, to different areas of the blood vessel;
monitoring a heart rate of the patient before applying the nerve modulating electrical field;
monitoring the heart rate upon applying the nerve modulating electrical field to each of the different areas of the blood vessel; and
assessing nerve density in the different areas based on the monitored heart rate;
wherein an increase in value of the heart rate corresponds to an increase in the nerve density.

7. The method of claim 6,
wherein applying the nerve modulating electrical field to different areas of the blood vessel includes moving the nerve modulating device to different axial positions along a length of the blood vessel and orienting the nerve modulating device toward different circumferential positions around a circumference of the blood vessel.

8. The method of claim 6, further comprising:
monitoring a systolic blood pressure (SysBP) of the patient before applying the nerve modulating electrical field; and
monitoring the SysBP upon applying the nerve modulating electrical field to the different areas of the blood vessel;
wherein assessing nerve density comprises calculating, for each of the different areas, an innervation index which is Delta HR*Delta SysBP, where Delta HR is a change in heart rate (HR) and Delta SysBP is a change in the SysBP, upon applying the nerve modulating electrical field.

9. The method of claim 8, further comprising:
generating an innervation index map of the innervation index as a function of the different areas of the blood vessel; and
superimposing the innervation index map with an anatomical image of the blood vessel to provide a superimposed map that shows varying levels of innervation indicative of different nerve densities, including one or more peak innervation levels, in the different areas of the blood vessel.

10. The method of claim 9, further comprising:
applying denervating energy to one or more target areas of the blood vessel based on the superimposed map, the one or more target areas having relatively higher levels of innervation indicative of relatively higher nerve densities.

11. The method of claim 10,
wherein the denervating energy is applied to the one or more target areas of the blood vessel that include the one or more peak innervation levels.

12. A method comprising:
introducing a catheter having an optical emission port into a blood vessel of a patient;
emitting a nerve modulating optical beam from the catheter through the optical emission port;
directing the optical beam to different areas of the blood vessel;
monitoring a parameter of afferent stimulation of the patient before emitting the optical beam;
monitoring the parameter of afferent stimulation upon directing the optical beam to each of the different areas of the blood vessel; and
assessing nerve density in the different areas based on the monitored parameter.

13. The method of claim 12,
wherein the parameter of afferent stimulation is related to at least one of inotropic effect or dromotropic effect of a heart of the patient.

14. The method of claim 12,
wherein the parameter of afferent stimulation is selected from the group consisting of a heart rate of the patient and a blood pressure of the blood vessel; and
wherein an increase in value of the parameter corresponds to an increase in the nerve density.

15. The method of claim 12,
wherein the optical beam is a low-intensity, pulsed infrared light beam.

16. The method of claim 12,
wherein directing the optical beam to different areas of the blood vessel includes moving the optical emission port to different axial positions along a length of the blood vessel and orienting the optical emission port toward different circumferential positions around a circumference of the blood vessel.

17. The method of claim 16,
wherein monitoring a parameter of afferent stimulation before applying the nerve modulating energy comprises monitoring a heart rate (HR) and a systolic blood pressure (SysBP) of the patient before emitting the optical beam; and
wherein monitoring the parameter of afferent stimulation upon applying the nerve modulating energy comprises monitoring the HR and the SysBP upon directing the optical beam to the different areas of the blood vessel;
wherein assessing nerve density comprises calculating, for each of the different areas, an innervation index which is Delta HR*Delta SysBP, where Delta HR is a change in the HR and Delta SysBP is a change in the SysBP, upon directing the optical beam.

18. The method of claim 17, further comprising:
generating an innervation index map of the innervation index as a function of the different areas of the blood vessel; and
superimposing the innervation index map with an anatomical image of the blood vessel to provide a superimposed map that shows varying levels of innervation indicative of different nerve densities, including one or more peak innervation levels, in the different areas of the blood vessel.

19. The method of claim 18, further comprising:
applying denervating energy to one or more target areas of the blood vessel based on the superimposed map, the one or more target areas having relatively higher levels of innervation indicative of relatively higher nerve densities.

20. A system comprising:
an implantable nerve modulating device adapted to be delivered to a renal vessel of a patient;
a nerve modulation module adapted to control the nerve modulating device to modulate renal nerves in different areas of the vessel;
a parameter monitoring module adapted to monitor one or more parameters of afferent stimulation of the patient; and
a nerve density assessment module adapted to assess nerve density of the different areas of the vessel based on the one or more monitored parameters.
